(19) United States
(12) Patent Application Publication
(10) Pub. No.: US 2026/0115314 A1
(43) Pub. Date: Apr. 30, 2026

(54) PREVENTION AND/OR TREATMENT OF CARDIAC DAMAGE

(71) Applicants: FUNDACIÓN UNIVERSIDAD FRANCISCO DE VITORIA, Pozuelo de Alarcón (ES); FUNDACIÓN PARA LA INVESTIGACIÓN BIOMÉDICA DEL HOSPITAL UNIVERSITARIO RAMÓN Y CAJAL, Madrid (ES); UNIVERSIDAD COMPLUTENSE DE MADRID, MADRID (ES)

(72) Inventors: Carlos ZARAGOZA SÁNCHEZ, Madrid (ES); Laura TESORO SANTOS, Pozuelo de Alarcón (ES); Laura BOTANA VEGUILLAS, Pozuelo de Alarcón (ES); Rafael RAMÍREZ-CARRACEDO, Pozuelo de Alarcón (ES); Ignacio HERNÁNDEZ NAVARRO, Pozuelo de Alarcón (ES); Javier DÍEZ-MATA, Pozuelo de Alarcón (ES); Marco FILICE, Madrid (ES); Karina OVEJERO-PAREDES, Madrid (ES)

(21) Appl. No.: 18/877,831

(22) PCT Filed: Jun. 23, 2022
(86) PCT No.: PCT/EP2022/067242

§ 371 Date: Dec. 20, 2024

(51) Int. Cl. A61K 47/69; A61K 9/51; A61K 47/62; A61K 49/00; A61K 51/08; A61K 51/12; A61P 9/10; A61P 29/00

(52) U.S. Cl. CPC A61K 47/6929; A61K 9/5146; A61K 47/62; A61K 49/0093; A61K 51/088; A61K 51/1244; A61P 9/10; A61P 29/00; A61K 2123/00

(57) ABSTRACT

The present invention refers to a peptide, comprising or consisting of SEQ ID NO: 1 (CAYMTMKIRN), for use as a medicament, preferably in the prevention and/or treatment of cardiac damage arising after ischemia followed by reperfusion, or in the prevention and/or treatment of the inflammatory response following acute myocardial infarction. In a preferred embodiment, the peptide is conjugated with a nanoparticle.

### FIELD OF THE INVENTION

The present invention refers to the medical field. Particularly, the present invention refers to a peptide, comprising or consisting of SEQ ID NO: 1 (CAYMTMKIRN), for use as a medicament, preferably in the prevention and/or treatment of cardiac damage arising after ischemia followed by reperfusion, or in the prevention and/or treatment of the inflammatory response following acute myocardial infarction. In a preferred embodiment, the peptide is conjugated with a nanoparticle.

### STATE OF THE ART

Cardiovascular diseases, including acute myocardial infarction (AMI), are the most frequent cause of death in the world. AMI remains the commonest origin of heart failure (HF), which conditions long-term life quality and may end up to the death of the patient.

Following an ischemic event, the lack of oxygen and nutrients in the tissue triggers the activation of inflammation. First, necrotic cardiomyocytes release danger signals that stimulate innate immune signalling, then cell mobilization begins to the damaged area and finally, anti-inflammatory signalling led to replacement by a fibrotic scar formation. Even though inflammation is a critical component of tissue healing, currently more contributions point towards a prolonged inflammatory response can compromise myocardial structure and function due to an adverse left ventricle remodelling, causing HF.

There is an unmet medical need of finding reliable strategies focused on the prevention and/or treatment of cardiac damage arising after ischemia followed by reperfusion, or in the prevention and/or treatment of the inflammatory response following acute myocardial infarction. The present invention is focused on solving this problem and an innovative therapeutic strategy is herein provided.

### DESCRIPTION OF THE INVENTION

### Brief Description of the Invention

The present invention refers to a peptide, comprising or consisting of SEQ ID NO: 1 (CAYMTMKIRN), for use as a medicament, preferably in the prevention and/or treatment of cardiac damage arising after ischemia followed by reperfusion, or in the prevention and/or treatment of the inflammatory response following acute myocardial infarction. In a preferred embodiment, the peptide is conjugated with a nanoparticle.

It is common general knowledge that early response after AMI is crucial for adequate resolution of inflammation and prevention of adverse cardiac remodelling. Macrophages orchestrate the overall inflammatory process from initiation to resolution, making them crucial cells in the development and progression of AMI.

The inventors of the present invention propose the use of a peptide analogue of Interleukin-10 (IL-10), with sequence SEQ ID NO: 1 (CAYMTMKIRN), preferably a nanoparticle conjugated with the peptide (NIL10), to target the IL-10-receptor in subjects exposed to ischemia/reperfusion (IR). Administration of 10 mg/kg NIL10 24 hours after IR in mice, induced a functional recovery of the left ventricle ejection fraction (LVEF) by days 3 and 7 after IR, when compared to the levels found in mice injected with NIL10SC, a nanoparticle conjugated with the same peptide in a scrambled orientation, in which the number of inflammatory foci and cardiac fibrosis was increased. In the same way, in mice deficient for IL10 subjected to IR, NIL10 also induced a functional recovery of the LVEF, while in IL10 receptor null mice, NIL10 did not show signs of improvement. The same level of cardioprotection was obtained by administration of 1 mg/kg NIL10 in pigs under IR, as detected by a reduction in the LVEF and the area of necrosis. To test whether NIL10 may participate in macrophage polarization, the inventors of the present invention injected 1 mg/kg NIL10 in pigs subjected to IR, in which M2 macrophage populations were significantly increased by day 3 post IR, when compared to the levels found in pigs injected with the same dose of NIL10SC, detecting that anti-inflammatory cytokines, including IL4, IL7, IL10, IL13, IL16 and IL27 resulted increased in mice and in pigs injected with NIL10. To test for the downstream effect of NIL10, the inventors found in both species that NIL10 induced activation of the STAT3 signalling pathway, and STAT3-dependent inhibition of NF-kB nuclear translocation, as detected accumulation of nuclear p65 by incubating RAW 247 macrophages with NIL10 and STATTIC, a pharmacological inhibitor of STAT3.

In conclusion, administration of NIL10 induces cardiac protection in mice and pigs subjected to IR, by inducing polarization towards M2 resolving macrophages, at least through inhibition of STAT3-induced proinflammatory inhibition of NF-kB nuclear translocation.

Thus, the first embodiment of the present invention refers to a peptide comprising or consisting of SEQ ID NO: 1 (CAYMTMKIRN) (hereinafter “peptide of the invention”) for use as a medicament.

In a preferred embodiment, the present invention refers to the peptide of the invention for use in the prevention and/or treatment of cardiac damage arising after ischemia followed by reperfusion or in the prevention and/or treatment of the inflammatory response following acute myocardial infarction.

In a preferred embodiment, the peptide of the invention is conjugated with a nanoparticle.

The nanoparticles used according to the present invention have a least 3 characteristics:
1. They have a PEG coating that improves the colloidal properties in blood stream, reduces the opsonization and, thus, enhances the pharmacodynamic and pharmacokinetic of the nanoparticles.
2. They comprise a dye, for instance rhodamine, which allows the visualization of the nanoparticle by in vivo fluorescence molecular imaging.
3. They comprise a chemical compound, such as gadolinium, which allows the detection of the nanoparticle in vivo, non-invasively, by nuclear magnetic resonance molecular imaging. This confers to the compound of the invention the possibility to be used in a patient without the need for an invasive procedure (cardiac biopsy or other invasive treatment) to visualise its effect with enhanced resolution.

It is important to note that, in order to obtain the required physical properties of the nanoparticles of the invention, and in order to reach the optimal colloidal characteristics that are a crucial parameter for their in vivo application in the prevention and/or treatment of cardiac damage, the size and polydispersity (PDI) of nanoparticles must be improved. To this end, such as it is indicated in Example 1.9, after the synthesis and characterization of the nanoparticles, an emulsification-mediated post processing was carried out. After that, a specific characterization was carried out to confirm the desired size (comprised between 100 and 150 nm in hydrodynamic size) and PDI (lower than 0.3) values reduction. Moreover, in order to finely modulate the biological activity, it is important also to control the density of the ligand (in term of ratio between peptide per lipid amounts) exposed on nanoparticles' surface. This aspect was elucidated by HLPC analysis. As general consideration, depending on the application and on the ligand class, for biomedical application this mg_{ligand}/mg_{lipid} ratio is expected to be comprised between 1:5 (highest ligand density) and 1:15 (lowest ligand density), being 1:10 the desired value. In this case, after HPLC characterization, the mg_{peptide}/mg_{lipid} ratio was estimated as 1:8 and 1:9 for NIL10 and NIL10SC, respectively.

The second embodiment of the present invention refers to a nanoparticle (hereinafter “nanoparticle of the invention”) conjugated with a peptide comprising or consisting of SEQ ID NO: 1 (CAYMTMKIRN).

The third embodiment of the present invention refers to the nanoparticle of the invention for use as a medicament.

In a preferred embodiment, the present invention refers to the nanoparticle of the invention for use in the prevention and/or treatment of cardiac damage arising after ischemia followed by reperfusion or in the prevention and/or treatment of the inflammatory response following acute myocardial infarction.

The fourth embodiment of the present invention refers to a pharmaceutical composition which comprises a peptide comprising or consisting of SEQ ID NO: 1, or a nanoparticle conjugated with the peptide and, optionally, pharmaceutically acceptable excipients or carriers.

The fifth embodiment of the present invention refers to a nanoparticle conjugated with a peptide consisting of SEQ ID NO: 1, characterized in that the nanoparticle size is comprised between 100 and 150 nm, the Polydispersity Index (PDI) is lower than 0.3 and the ratio between the total amount of the peptide and the total amount of lipids in the nanoparticle is comprised within 1:5 and 1:15. In other words, there would be 1 peptide for every 5 to 15 lipids in the nanoparticle.

In a preferred embodiment, the nanoparticle conjugated with a peptide consisting of SEQ ID NO: 1 comprises a PEG coating and maleimide linking point, to enhance the pharmacokinetic properties and to enable the linking of SEQ ID NO: 1 peptide, respectively.

In a preferred embodiment, the nanoparticle conjugated with a peptide consisting of SEQ ID NO: 1 comprises a dye which allows the visualization of the nanoparticle by fluorescence imaging.

In a preferred embodiment, the nanoparticle conjugated with a peptide consisting of SEQ ID NO: 1 comprises a chemical compound or radioisotopes which allows the detection of the nanoparticle by in vivo molecular imaging techniques.

The sixth embodiment of the present invention refers to the nanoparticle conjugated with a peptide consisting of SEQ ID NO: 1, as defined in any of the above embodiments, for use as a medicament, for use in the prevention and/or treatment of cardiac damage arising after ischemia followed by reperfusion, or for use in the prevention and/or treatment of the inflammatory response following acute myocardial infarction.

The seventh embodiment of the present invention refers to a pharmaceutical composition comprising the nanoparticle conjugated with a peptide consisting of SEQ ID NO: 1 of any of the above embodiments, and, optionally, pharmaceutically acceptable excipients or carriers.

The eight embodiment of the present invention refers to the above defined pharmaceutical composition for use in the prevention and/or treatment of cardiac damage arising after ischemia followed by reperfusion or for use in the prevention and/or treatment of the inflammatory response following acute myocardial infarction.

Alternatively, the present invention refers to a method for the prevention and/or treatment of cardiac damage arising after ischemia followed by reperfusion, or for the prevention and/or treatment of the inflammatory response following acute myocardial infarction, which comprises the administration of a therapeutically effective dose or amount of the peptide or nanoparticle of the invention.

For the purpose of the present invention the following terms are defined:
The term “comprising” is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.
By “therapeutically effective dose or amount” is intended an amount that, when administered as described herein, brings about a positive therapeutic response in the patient. The exact amount required will vary from subject to subject, depending on (non-exhaustive list): the species, age, general condition of the subject, the severity of the condition being treated or the mode of administration. An appropriate “effective” amount in any individual case may be determined by one of ordinary skill in the art using routine experimentation, based upon the information provided herein.
The term “nanoparticle”, as used in the present invention, refers to any particle that has at least one of its dimensions smaller than about 1000 nm. The person skilled in the art is able to obtain nanoparticles according to the needs. The diameter of the nanoparticles can be about 5 nm or 10 nm or 20 nm or 30 nm or 40 nm or 50 nm or 60 nm or 70 nm or 80 nm or 90 nm or 100 nm or 200 nm or even larger.

### DESCRIPTION OF THE FIGURES

FIG. 1. NIL10 binds to IL-10R in RAW 264.7 cells. A. Nanoparticle composition of NIL10 and NIL10SC. B. Z-potential, longitudinal and transversal relaxivities of NIL10 and NIL10SC nanoparticles. C. Bright field and confocal microscopy sections of RAW 264.7 macrophages, detecting rhodamine (red) containing NIL10 nanoparticles. D,E Colocalization of NIL10 (D) (rhodamine, red) or NIL10SC (E) with IL-10R (FICT, green) in confocal microscopy sections of RAW 264.7 cells incubated with NIL10 and anti-IL-10R specific antibody. Merged panel show co-localization of both signals. Nuclei were stained with Hoechst. Right panel: colocalization analysis with Image J co-localization plugin software, in which white dots correspond to co-localization (N=3).

FIG. 2. Dose-effect of NIL10 on animal survival and organ biodistribution. A. Kaplan Meier curve showing the percentage of survival of healthy pigs injected with the doses indicated of NIL10 (N=10 mice/condition). B. Confocal microscopy detection of NIL10 and NIL10SC in heart, liver, kidney pancreas, spleen and lung sections of healthy animals (N=10 mice/group).

FIG. 3. NIL10 induces cardiac protection in mice subjected to IR. A. Outline of the proceedings performed. B. Representative electrocardiogram, showing ST-elevation after LAD occlusion. C. Left ventricle ejection fraction (LVEF) of wild type mouse hearts at the times indicated after IR and injected with NIL10 or NIL10SC as in A. N=10 mice/group. Results expressed as mean±SD. *P<0.03 NIL10 vs NIL10SC day 7. #P<0.05 NIL10 vs NIL10SC day 3. D. LVEF in IL-10 knockout mice. N=10 mice/group. Results expressed as mean±SD. *P<0.01 NIL10 vs NIL10SC day 7. #P<0.05 NIL10 vs NIL10SC day 3. E. LVEF in IL-10 receptor knockout mice. N=10 mice/group. Results expressed as mean±SD.

FIG. 4. Hematoxylin and Eosin staining of mouse hearts. Heart sections of mice subjected to IR and injected with NIL10 or NIL10SC and collected by days 3 (A) or 7 (B) after IR. N=10 mice/group. From left to right: 2×, 20×, 40×. *P<0.05 D3; **P<0.01D7.

FIG. 5. NIL10 reduce heart fibrosis in response to IR. A. Representative immunoblot of MMP9 from healthy (H) or necrotic (N) areas of mouse hearts after 7 days of IR and injected with NIL10 or NIL10SC. N=10 mice/group. Mean±SD. *P<0.001 NIL10 vs NIL10SC necrotic areas. B. Masson Trichrome staining of heart sections from mice injected with NIL10 or NIL10SC, after 3- or 7-days post IR (N=10 mice/group). *P<0.05 D3; **P<0.03 D7.

FIG. 6. NIL10 induces cardiac protection in pigs subjected to IR. A. Evans blue/TTC staining (see methods for details) of pig heart sections (from apex to base) after 7 days of IR, showing healthy tissue (blue), the area at risk (red) and the necrotic areas (white). B. Measurement of necrotic areas of the hearts, represented as a percentage respect to the area at risk (Mean±SD. *P<0.001 NIL10 vs NIL10SC, at day 7 post IR). C. Left ventricle ejection fraction of hearts from pigs injected with NIL10 or NIL10SC after 7 days of IR (Mean±SD. *P<0.001 NIL10 vs NIL10SC). D. Hematoxylin and Eosin staining of healthy and necrotic heart sections from pigs injected with NIL10 or NIL10SC after 7 days of IR. E. Masson Trichrome staining of the same hearts as in D. (N=10 pigs/group).

FIG. 7. NIL10 induces M2 macrophage polarization in the hearts of mice subjected to IR. A. Flow cytometry analysis of macrophage populations in mouse hearts after 3 and 7 days of IR. The percentage of M2 CD68+/CD206+ population were selected from the necrotic and at-risk areas of the hearts. B. Gating strategy used to identify macrophage-cell subsets in the health and infarcted mouse heart. A sequential getting strategy was first used to identify M2 population expressing specific macrophage marker CD68, followed by the identification of the population with overlapping expression patterns (CD68/CD206) (N=10 mice/group). Differences between groups were compared using one-way ANOVA. **P<0.01. ***P<0.001. ****P<0.0001.

FIG. 8. NIL10 induces the expression of resolving cytokines. A. Cytokine array used in the study. B. Clustered heat map of the differentially expressed cytokines in mice. C. Clustered heat map of the differentially expressed cytokines in pigs. Red arrows indicated anti-inflammatory cytokines.

FIG. 9. NIL10 induces phosphorylation of IL10RA and STAT3. A. Immunoblot detection of P-IL10-RA (upper), and total IL-10-RA in the healthy (H) and necrotic (N) areas of mouse hearts at day 7 after IR (P<0.04 NIL10 vs NIL10SC necrotic areas). B. Immunoblot detection of P-STAT3, and total STAT3 in the same protein extracts. N=10 mice/group. Results expressed as Mean±SD (P<0.05 NIL10 vs NIL10SC necrotic areas).

FIG. 10. NIL10 induces phosphorylation of IL10RA and STAT3. A. Immunoblot detection of P-IL10-RA (upper), and total IL10-RA in the healthy (H) and necrotic (N) areas of pig hearts at day 7 after IR (P<0.05 NIL10 vs NIL10SC necrotic areas). B. Immunoblot detection of P-STAT3, and total STAT3 in the same protein extracts. N=10 pigs/group. Results expressed as Mean±SD (P<0.005 NIL10 vs NIL10SC necrotic areas).

FIG. 11. NIL10 prevents nuclear translocation of NF-KB trough STAT3 activation in RAW 264.7 macrophages. A. Confocal microscopy detection of NF-κB (p65) in RAW 264.7 cells stimulated with 500 μM LPS or in combination with NIL10. Nuclei were stained with DAPI. B. Immunoblot detection of P-IκB-α, or total IκB-α in RAW 264.7 cells treated with NIL10 or NIL10SC. (N=3, results expressed as Mean±SD. *P<0.05 LPS/NIL10 vs LPS/NIL10SC). C. Confocal microscopy detection as in A, in which STATTIC, a pharmacological inhibitor of STAT3 was incubated. Arrows point nuclear or cytoplasmic localization of p65. Lower panels: immunoblot detection of iNOS in cells as in C. (N=3, results expressed as Mean±SD. *P<0.03 LPS vs LPS/NIL10 **P<0.05 LPS/STATTIC vs LPS/NIL10SC/STATTIC. ***P<0.001 NIL10/STATIC vs LPS/NIL10/STATIC).

### DETAILED DESCRIPTION OF THE INVENTION

The present invention is illustrated by means of the Examples set below without the intention of limiting its scope of protection.

### Example 1. Material and Methods

### Example 1.1. Reagents and Equipment

Hematoxylin-eosin (HE), Trichrome Masson staining reagents, Triphenyl tetrazolium chloride (TTC), Evans Blue and fetal bovine serum (FBS) were from Merck (St Louis, MO, USA). Horseradish peroxidase (HRP)-conjugated anti-mouse secondary antibody and liquid 3,3′-diaminobenzidine (DAB) substrate were from Dako (Santa Clara, CA). Anti-IL10RA antibody (ab225820) was from Abcam (Cambridge, UK), Proteome Profiler Mouse Cytokine Array Kit (ARY006) and Proteome Profiler Human Cytokine Array Kit (ARY005B) was from R&D Systems (Minneapolis, MN), ketamine was from Pfizer (New York, NY, US), isoflurane was from Abbvie (North Chicago, IL, US), propofol was from Fresenius (Bad Homburg, Germany), fentanyl was from Kern Pharma (Madrid, Spain), diazepam was from Roche (Basel, Switzerland), and the amiodarone was from Sanofi Aventis (Gentilly, France).

The following is a list of the most common equipment used for this investigation: 5415R Refrigerated Centrifuge was from Eppendorf (Hamburg, Germany). The chemiluminescence imaging system Fusion Solo-S and the image analysis software Fusion-Capt were from Vilber-Lourmat (Eberhardzell, Germany). TCS-SP5 Confocal Microscope was from Leica (Wetzlar, Germany). The microplate reader was from Biotek (Winooski, VT). NanoDrop One Spectrophotometer was from Thermo Scientific (Waltham, MA). Guiding catheters, angioplasty balloons and catheter introducers were from Cordis (Miami, FL). Diagnostic and steerable guidewires were from Boston Scientifics (Malborough, MA). The balloon inflation devices and midazolam were from B.Braun (Melsungen, Germany).

### Example 1.2. Animal Model of Coronary Ischemia/Reperfusion

All the surgical procedures were performed in the Experimental Surgery Department of the Hospital Universitario La Paz (Madrid, Spain) in conforming to the Guide for the Care and Use of Laboratory Animals published by the US National Institutes of Health (NIH Publication No. 85-23, revised 1985), and the Animal Welfare Ethics Committee and complied with the EU Directive on experimental animals (63/2010 EU) and related Spanish legislation (RD 53/2013).

20 male Yorkshire pigs (30±4 kg) were housed 1 week preceding the surgery to avoid unease or stress associated with the new environment. Prior to the surgical intervention, animals were anesthetized with intramuscular ketamine 10 mg/kg and midazolam 0.5 mg/kg.

Anesthesia was induced by inhaled sevoflurane and maintained with continuous infusion of propofol 2 mL/kg/h, fentanyl 50 μg/kg/h, and diazepam 10 μg/kg/h. After intubation and mechanical ventilation with 100% oxygen saturation, 5000 IU of heparin and amiodarone 2 mg/kg/h was administered to avoid blood clotting of catheters and malignant cardiac arrhythmias, respectively. Preceding the complete occlusion, hearts were submitted to ischemic preconditioning by blocking the LAD for short periods (1, 3, and 5 minutes each). Ischemia/reperfusion was produced by occluding LAD for 45 minutes using a JL3 6F catheter and an angioplasty balloon. The complete myocardial ischemia was confirmed by ST-segment elevation. Then, the balloon was removed to reopen the artery. Animals received NIL10 or NIL10SC (0.1 mg/kg) 24 h hours post-ischemia. Blood was extract before and after the procedure, 3 days post-ischemia and at final point. After 7 days of reperfusion, animals were sacrificed to extract the heart, spleen, kidney, lung, liver and pancreas. This samples were included in 4% formalin and other samples of the heart were immediately frozen at −80° C. to protein analyses.

20 male twelve-week-old wild type, IL10 knock-out and IL10RA knock-out mice C57/BL6 were anesthetized by inhaled 3% sevoflurane and oxygen with a flow rate of 0.4 L/min until loss of righting reflex. Then, endotracheal intubation was perform using an intubation cannula in order to carry out artificial ventilation (tidal volume: 260 μL/stroke, ventilation rate: 130 strokes per minute). The fourth left intercostal space was opened and widened using chest mice retractors. Left ventricle was exposed and LAD was occluded for 30 min close by using a 6-0 silk suture and 1 mm tube. Reperfusion was performed by ligation release. After the procedure, the chest was closed, negative pressure restored, and the skin sutured. At the reperfusion time of 24 hours, NIL10 was venous-administrated. Animals were sacrificed at day 3, 7 or 21 to extract the organs and process the samples. NIL10SC-treated animals were included in the assays as control, in which the same procedure was performed.

### Example 1.3. Echocardiography

Vivid Q ultrasound system from GE Healthcare (General Electric, Chicago, IL, USA), equipped with a 1.9-4 MHz scan head was used to determine LV function using a. Parasternal long and short-axis-view images of the heart were taken prior to the surgery, at the end of ischemia, and at the endpoint to determine LV function worsening and recovery. The parameters studied using the on-site software cardiac package were: systolic and diastolic interventricular septum thickness (IVS), systolic and diastolic left-ventricle internal diameter (LVID), systolic and diastolic left-ventricle posterior wall thickness (LVPW), left-ventricle ejection fraction (EF), left ventricle shortening fraction (FS), heart rate (HR), cardiac output (CO) and left ventricle ejection fraction (LVEF). Data acquisition and analysis were performed by the same operator to avoid the inter-observer error.

### Example 1.4. Histology

Blue/TTC staining in pigs: 7 days after the surgery, the LAD was reoccluded in the same location as day 0, following the same method described before. Then, 200 mL of 5% Evans Blue solution was injected using a 5F fenestrated Pigtail catheter within the left ventricle, to distribute the compound across the entire cardiovascular system, excepting the blood-deprived area of the heart. After 2 minutes, the animal was sacrificed, and the heart extracted to be frozen at −20° C. for 24 hours. The next day, hearts were cut into 0.8 cm slices and incubated in 1% TTC for 20 minutes at 37° C. and next in 10% Paraformaldehyde solution. The necrotic area was relativized to the area at risk to avoid inter-experiment variability concerning the ischemic area.

Heart morphology was visualized by Hematoxylin/Eosin staining and collagen deposition was detected by Masson's trichrome staining in mice and pigs.

### Example 1.5. Confocal Microscopy

Paraffin embedded 0.5 μm heart sections were incubated with anti-CD68 and anti-CD206 (diluted 1:500 in PBS 1.5% BSA) primary antibody overnight at 4 C. Slides were washed three times with PBS and mounted in PBS media, containing Hoechst for nuclei visualization. Images were taken using a Leica TCS SP5 confocal microscope. At least three different fields per condition were obtained.

### Example 1.6. Immunoblotting

Proteins from healthy and infarcted area of the heart of pigs and mice were extracted to measure MMP-9, IL10-RA, PHOSPHO-IL10-RA, STAT3 and PHOSPHO-STAT3 expression. 20 μg of total protein were loaded into 10%-7.5% polyacrylamide gels. After the electrophoresis, proteins were transferred to PVDF membranes and blocked with 3% BSA in T-TBS. Membranes were incubated for 1 hour with primary antibody 1:1000 and anti-Rabbit HRP-conjugated secondary antibody 1:3000. Then, protein bands were visualized by chemiluminescence and studied using image analysis software.

### Example 1.7. Peptide and Nanoprobe Composition

Peptide IT9302 (SEQ ID NO: 1):
CAYMTMKIRN
Peptide IT9302-scrambled:
CANYRMITKM

### Example 1.8. Nanoparticles Preparation

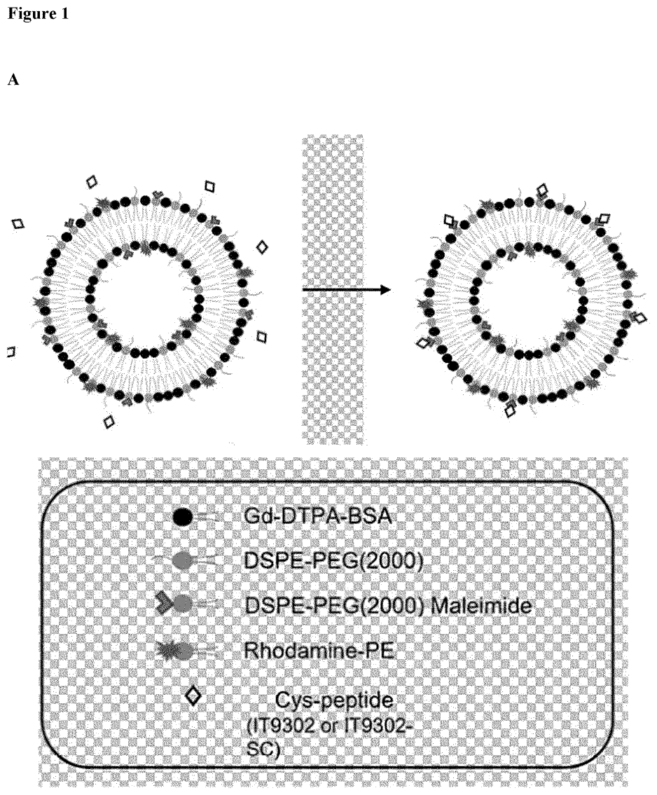

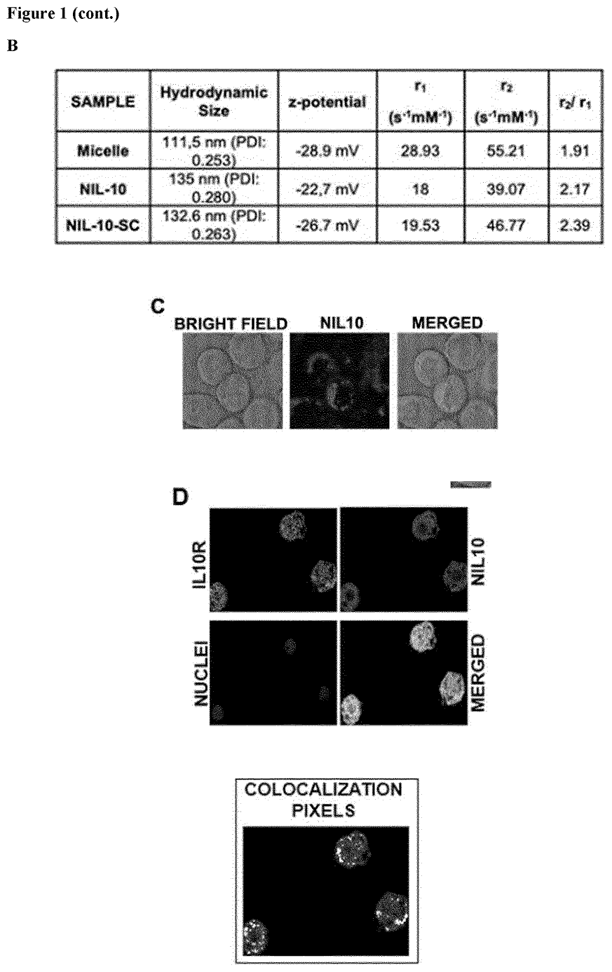

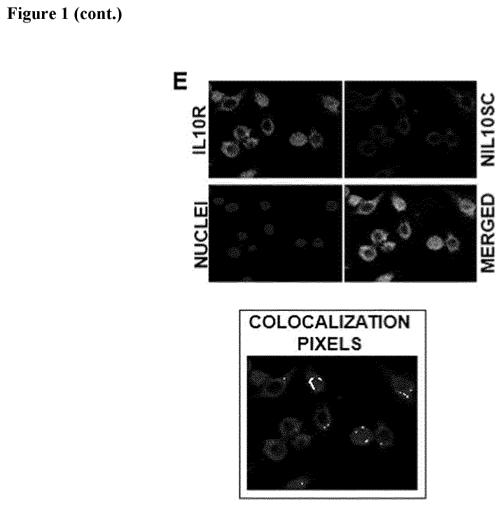

The paramagnetic nanoparticles were prepared by the lipid film hydration method. In brief, a film was prepared by rotary evaporation of Gd-DTPA-bis (GdDTPA-BSA), 1,2-distearoyl-sn-glycero-3-phosphoethanolamine-N-[methoxy (polyethylene glycol)-2000] (DSPE-PEG2000), DSPE-PEG2000-maleimide, and Rhodamine-PE, in a molar ratio 50:39:1:10, dissolved in a mixture of chloroform/methanol (4:1 v/v). The lipid film was hydrated with HEPES buffer, pH 6.7, and 150 mM NaCl, and the solution was rotated at 65° C. for 1 hour. NIL10 and the corresponding scrambled peptides NIL10SC were modified by adding a terminal cysteine residue (FIG. 1) to bind to the maleimide moiety at a molar ratio 1:40 (nanoparticle:peptide). Uncoupled peptides were separated with centrifuge concentrators of 100 kDa. Physical and chemical properties, including Zeta-potential, nanoparticle size (calculated as hydrated diameter with dynamic laser light scattering (DSL, Malvern Zetasizer)), nanoparticle morphology (visualized with transmission microscopy (TEM)), longitudinal and transverse relaxivities (Minispec mq relaxometer, Bruker), were calculated. Longitudinal relaxivities were evaluated by using inversion recovery sequences with 15 inversion times. Transverse relaxivities were calculated from spin-echo images and different echo times. In both cases, a series of images with different T1 and T2 weighting were generated. The r1 and r2 values were estimated from the slope of longitudinal and transverse relaxation rates vs Gd-DTPA amount per nanoparticle.

### Example 1.9. Improvement of Physical Properties of Peptide-Modified Nanoparticles

To improve the physical properties of peptide-modified nanoparticles and to reach the optimal colloidal characteristics that are required for their in vivo application (or the prevention and/or treatment of cardiac damage), the size and polydispersity (PDI) of nanoparticles must be improved. To this scope, an emulsification-mediated post processing was carried out. Therefore, after their synthesis and characterization, the nanoparticles described in Example 1.8 were homogenized three times with an Emulsiflex®-B15 (Avestin) emulsifier by setting the working pressure to 80 psi/5.5 bar. After that, the DLS characterization was carried out to confirm the desired size (comprised between 100 and 150 nm in hydrodynamic size) and PDI values reduction (lower than 0.3). Furthermore, even the peptide amount bound per mg of lipid was characterized by HLPC analysis and following this protocol: mobile phase 75% water, 25% acetonitrile and 0.1% trifluoroacetic acid, column Gemini® 5 μm NX-C18 110 Å column, room temperature, λ: 220 nm. The mg_{peptide}/mg_{lipid} ratio was estimated between 1:5 and 1:15, preferably 1:8 and 1:9 for NIL10 and NIL10SC, respectively.

### Example 1.10. Blood Collection and Plasma Isolation

Animal blood samples were collected in buffer Sodium-Citrate tubes (363086) and EDTA tubes (367861) from a retro-orbital bleed in mice or from femoral venous in pigs respectively and were from BD Vacutainer (Franklin Lakes, NJ, US). Plasma was isolated from blood spun at 400 g for 10 minutes. Plasma not used immediately was stored at −20° C.

### Example 1.11. Cytokine and Chemokine Determinations

We detected a total of 36 human cytokines, chemokines, and acute phase proteins simultaneously from the serum of pigs and mice at the times indicated, by using the Proteome Profiler Cytokine Array Kit (RD Systems, Minneapolis, MN) a membrane-based antibody array for the parallel determination of the relative levels of selected human cytokines and chemokines.

### Example 1.12. Single-Cell Suspension for Flow Cytometry

Whole mouse hearts were mashed in complete DMEM (10% FBS, penicillin/streptomycin) through a 100-μm cell strainer. ACK Lysing Buffer was added to the single-cell suspension and spun down at 350 g for 5 minutes. The ACK was washed out with 50 mL of washing buffer (1% FBS), and cells were incubated with 1:100 anti-EMMPRIN, anti-CD68 and anti-CD206 antibodies. All flow cytometry samples were assayed in a MACSQuant Analyzer Flow Cytometer and analyzed with the Graphpad Prism software package.

### Example 1.13. Statistical Analysis

All values were given as mean±S.D. Significance is reported at the 5% level. Whenever comparisons were made with a common control, significance of differences was tested by Dunnett's modification of the t test.

### Example 2. Results

### Example 2.1. NIL10 Improves Cardiac Function in Mice Subjected to IR

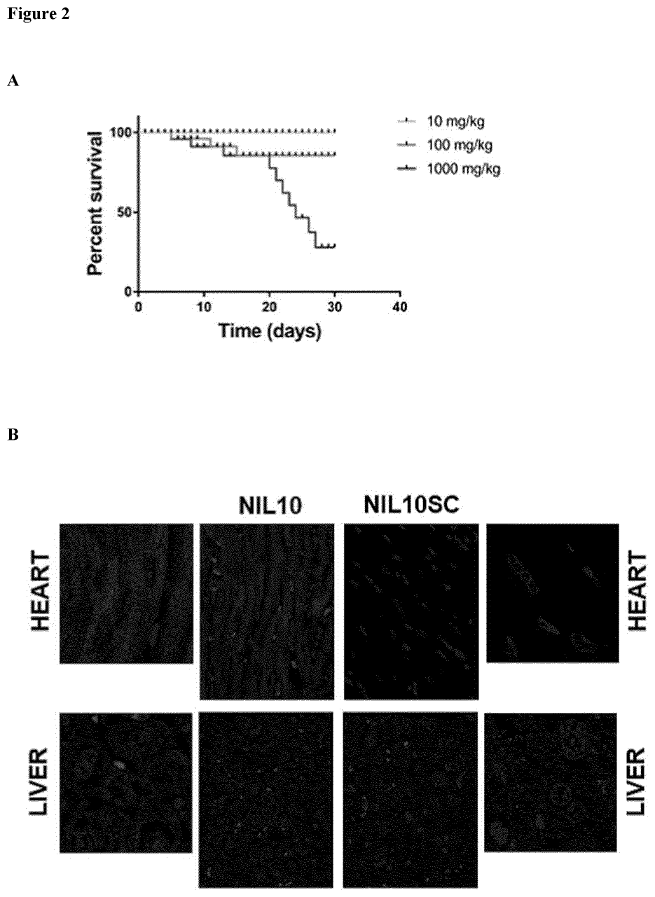

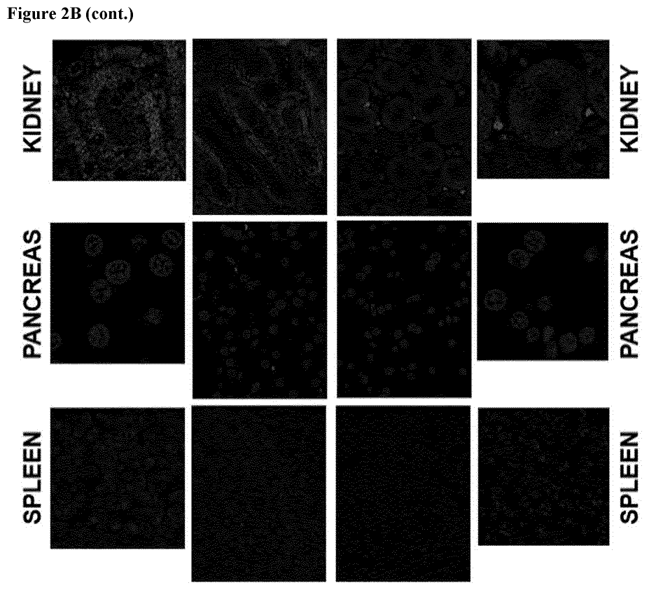

To study the specificity of nanoparticles, we first incubated NIL10 (FIG. 1A, B) in RAW 264.7 macrophages, detecting rhodamine-containing nanoparticles by confocal microscopy (FIG. 1C). Colocalization with the IL-10R (as detected by immunohistofluorescence (FITC, green) was positive in RAW 264.7 cells incubated with NIL10 (rhodamine, red. FIG. 1D), as detected by confocal microscopy (Merged panel, yellow and colocalization pixels panel), while no binding was found in cells incubated with NIL10SC, as NIL10SC is conjugated with a peptide which does not bind to IL10R (FIG. 1E). Kaplan Meier curves of healthy mice injected with 1, 10 and 100 mg/kg indicated no mortality linked to the lowest dose, whereas the dosages of 10 and 100 mg/kg were lethal for 20% and 75% of animals, respectively, after 30 days of testing (FIG. 2A).

To test for biodistribution, confocal microscopy visualization of NIL10 and NIL10SC in the heart, liver, kidney, pancreas, spleen and lungs of healthy animals revealed a faint accumulation of NIL10 in the heart, kidney and lungs (FIG. 2).

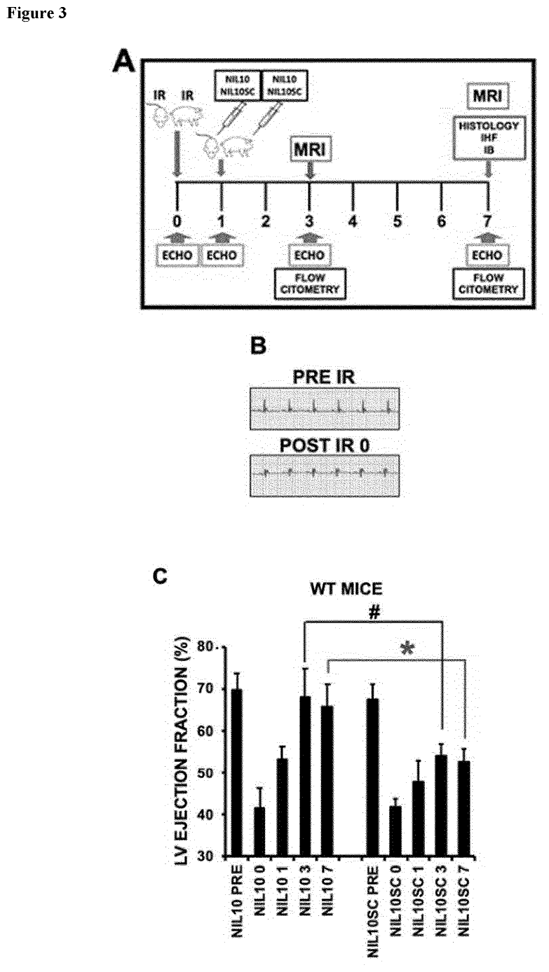

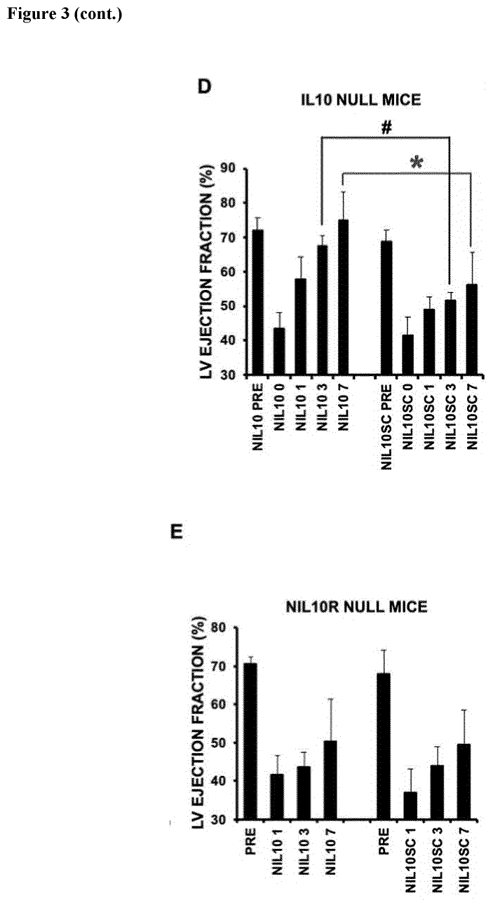

After selecting a dosage of 1 mg/kg, we proceeded to inject 1 mg/kg NIL10 or NIL10SC in mice 24 hours after IR (FIG. 3A, B), resulting that NIL10 improved the LVEF by days 3 and 7 after IR (41.5%±4.33 vs 68%±7.02 and 65.6%±5.11 respectively (FIG. 3C). Proof of the effect was obtained by performing the same assay in IL10 null mice, in which NIL10 also exhibited a significant degree of cardioprotection (FIG. 3D). By contrast, in IL-10 receptor deficient mice subjected to IR, NIL10 had no effect (FIG. 3E), suggesting that NIL10 induces cardiac protection at least, through activation of IL10 receptor signalling pathway.

### Example 2.2. Administration of NIL10 Reduces Necrosis and Fibrosis in Mouse Hearts Subjected to IR

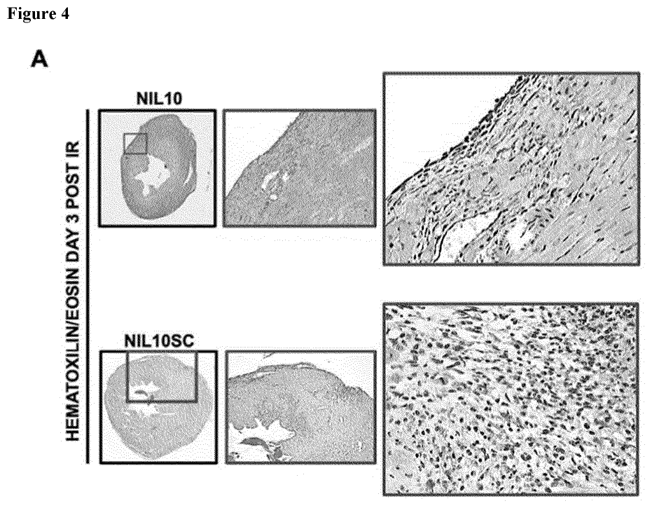

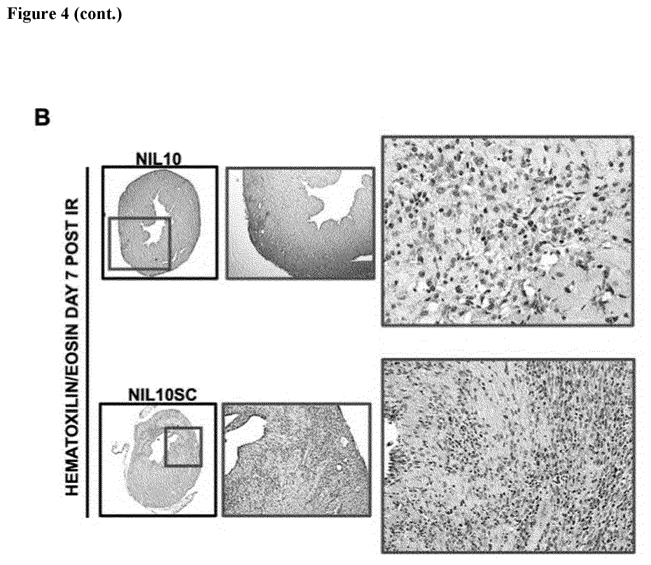

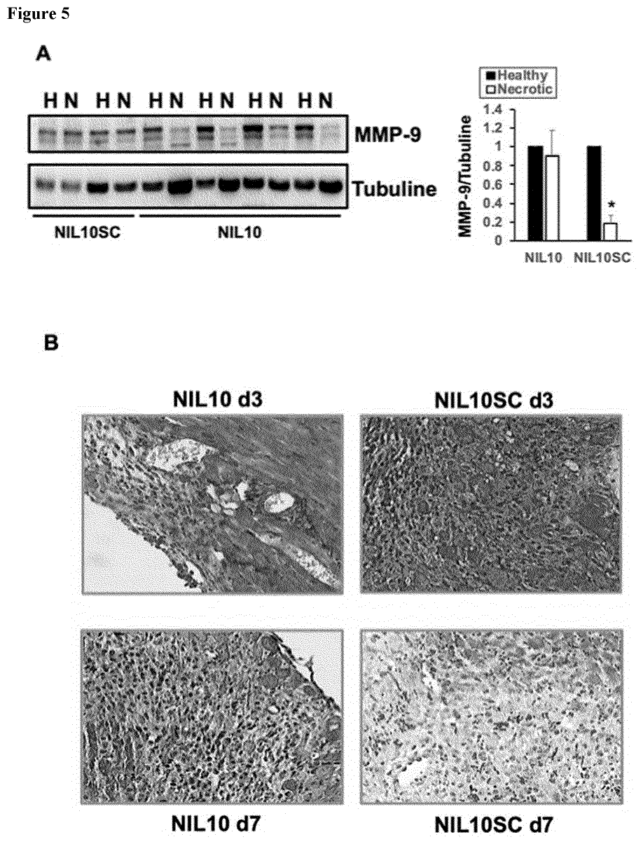

To shed light into the underlying causes by which NIL10 induces cardioprotection, Hematoxilin/Eosin (H/E) staining of heart sections isolated by days 3 and 7 after IR, showed a significant reduction in the inflammatory foci and the extension of myocardial necrosis in response to NIL10 (FIG. 4). Likewise, the levels of the necrosis marker, matrix metalloproteinase 9 (MMP9), were markedly reduced in the necrotic areas of NIL10-injected mice (FIG. 5A), including the extension of fibrosis, whereas in the NIL10SC group, fibrotic lesions were widespread in the hearts by day 7 after IR (FIG. 5B).

### Example 2.3. NIL10 Improves Cardiac Function in Pigs Subjected to IR

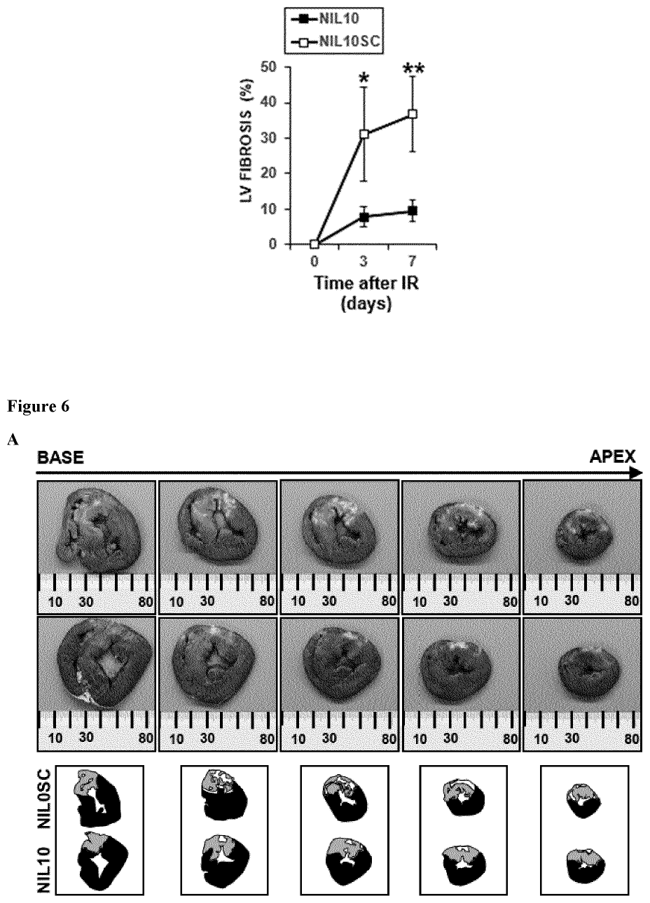

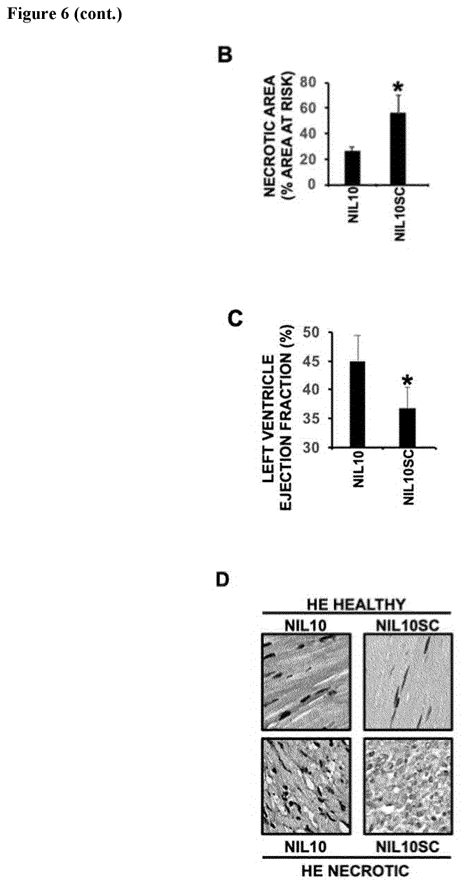

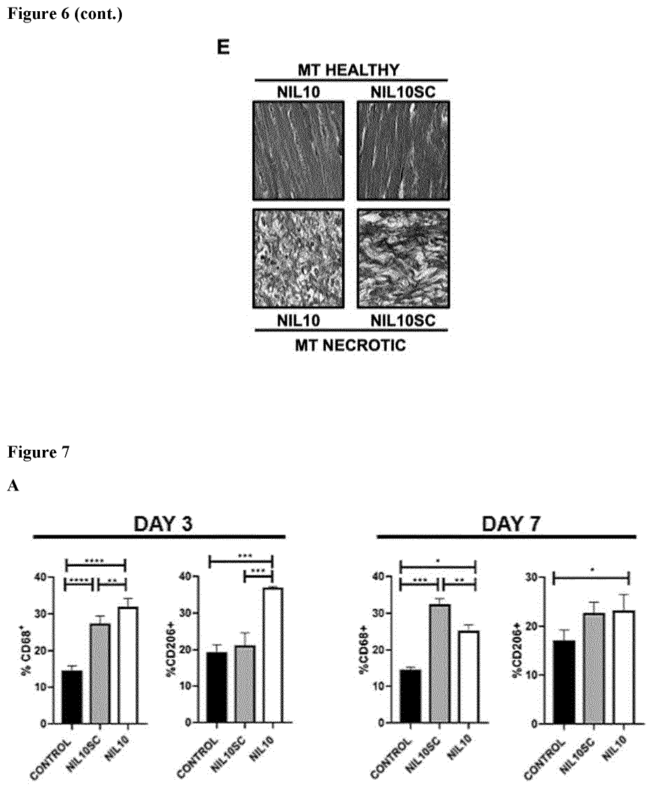

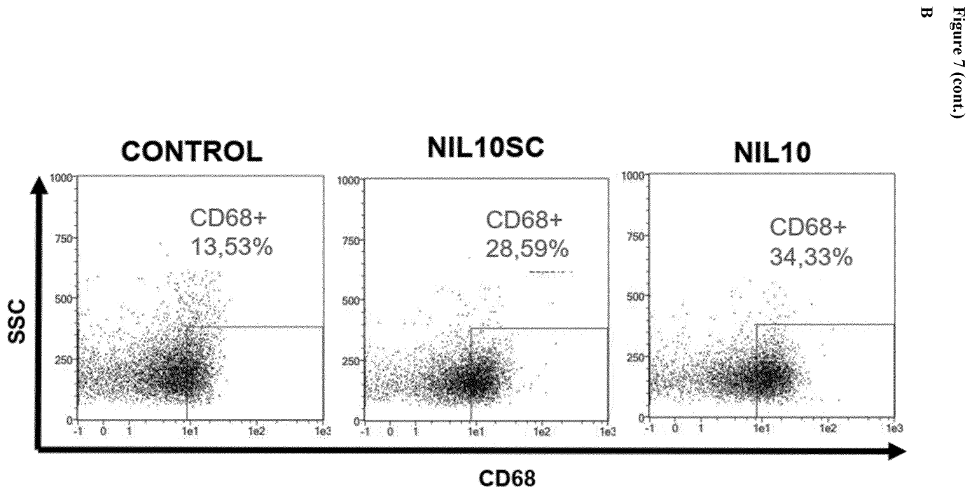

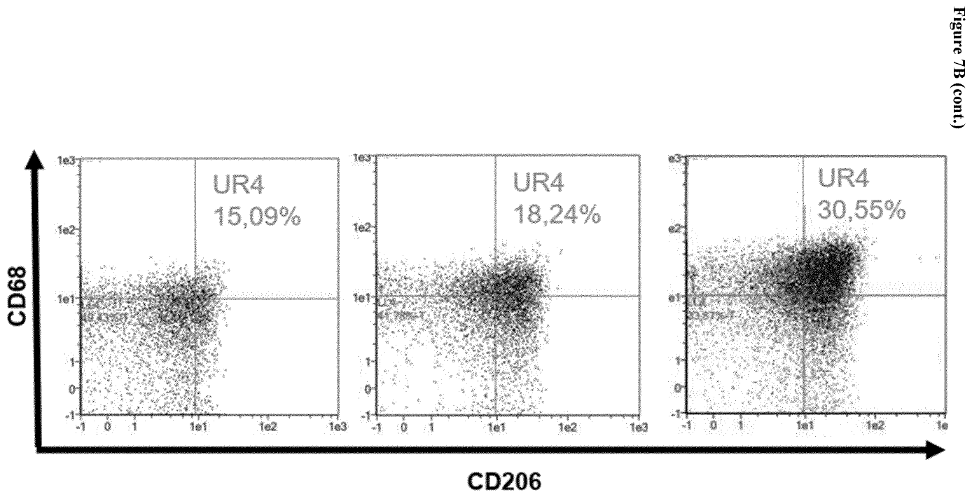

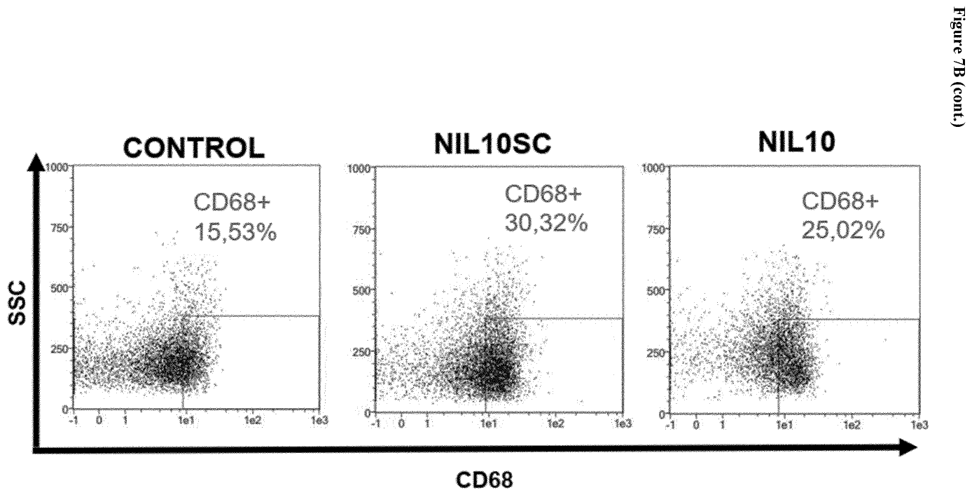

To test whether NIL10 may also induce cardiac protection in large animals, we injected 1 mg/kg NIL10 or NIL10SC into pigs subjected to IR by angioplasty balloon inflation of the LAD coronary artery (FIG. 3A). As shown by EvansBlue/TTC staining of left ventricle sections (FIG. 6A), after 7 days of IR we found a significant reduction of the necrotic area by 47% (NIL10SC 55±7.34 vs NIL10 26.48±4.32 (FIG. 7B), which contributes to explain the improvement in cardiac function as evidenced by the recovery of LVEF in animals injected with NIL10 (FIG. 6C). As in mice, HE and Masson's trichrome staining of heart sections from NIL10 injected pigs, indicated a marked reduction of myocardial necrosis (FIG. 6D) and fibrosis (FIG. 6E), when compared with the NIL10SC group.

### Example 2.4. Injection of NIL10 has an Impact on Macrophage Polarization

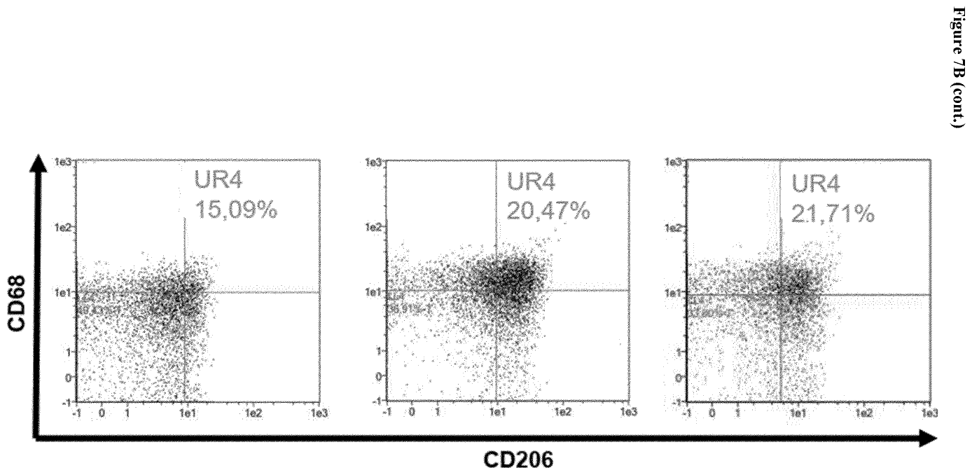

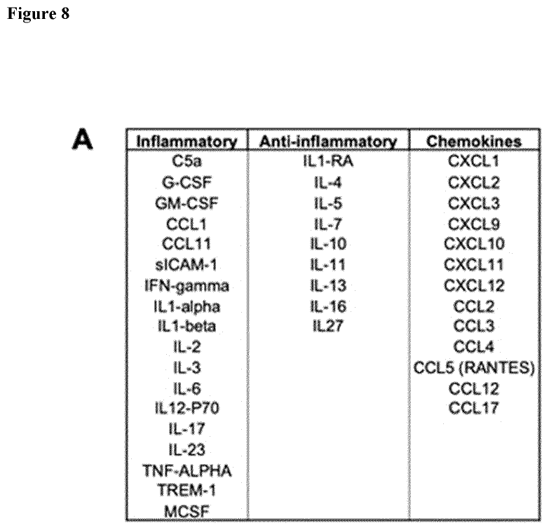

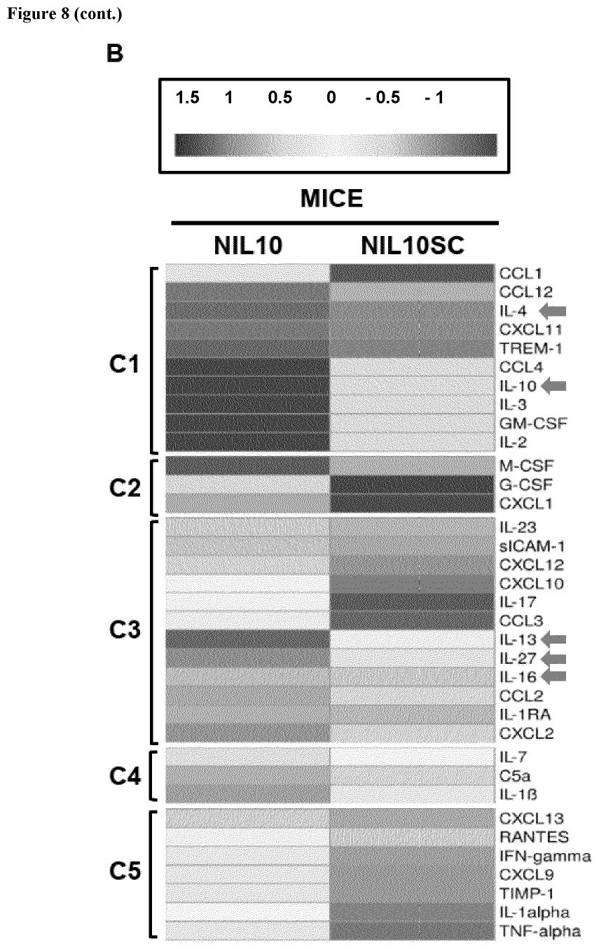

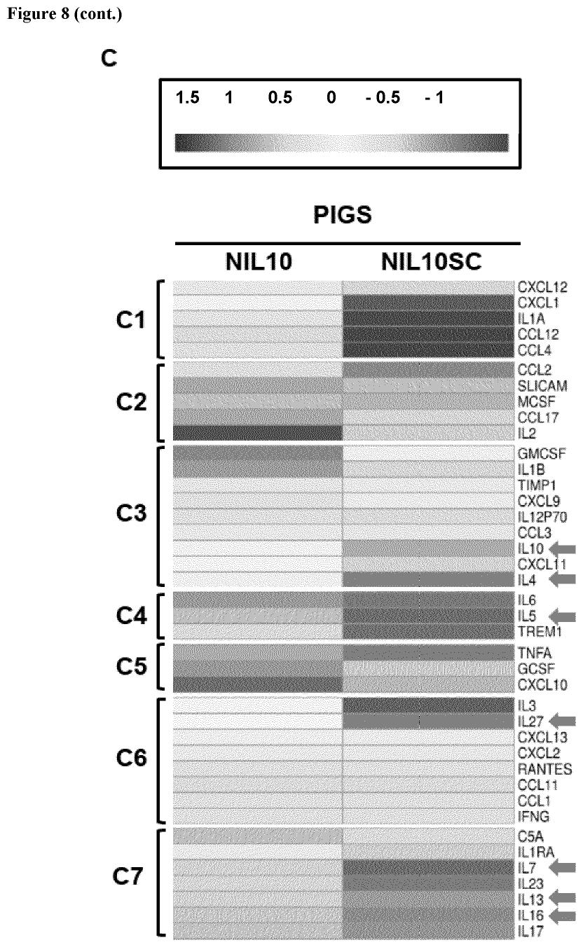

The observed differences in the number and severity of inflammatory foci led us to consider whether NIL10 might have an impact on macrophage polarization. Indeed, cell extracts obtained from the necrotic areas of mouse hearts indicated that after 3 days of IR, NIL10 promotes the presence of M2-resolving macrophages, while it takes 7 days to detect a similar effect when NIL10SC was injected (FIG. 7A-B), which may help to explain the reduction of inflammatory foci in animals injected with NIL10. We also assessed the level of 40 cytokines and chemokines in plasma collected from mice and pigs after 3 days of IR (FIG. 8A). We identified differences in the expression of a significant number of cytokines, highlighting the presence of 4 mouse (5 pig) expression clusters depending on whether the animals have been injected with NIL10 or NIL10SC, highlighting that in both species, we identified a marked increase in the expression of anti-inflammatory cytokines IL-4, -10, -13, -16, and -27, together with IL-5, and IL-7 in pigs, in response to NIL10 administration (FIG. 8BC). Taken together, these results suggest that NIL10 may induce cardiac protection by at least, macrophage polarization towards inflammation resolution.

### Example 2.5. NIL10 Induces Phosphorylation of STAT3 in Pigs Subjected to IR

Cardiac exposure to ischemia triggers activation of specific pro-inflammatory transcription factors, of which NF-κB plays a major role. Polarization of immune cells through an inflammatory resolving state implies the activation of specific signaling cascades, highlighting the contribution of IL-10-induced JNK signaling pathway that activates STAT3 transcriptional regulation of anti-inflammatory cascades, including prevention of NF-κB nuclear translocation.

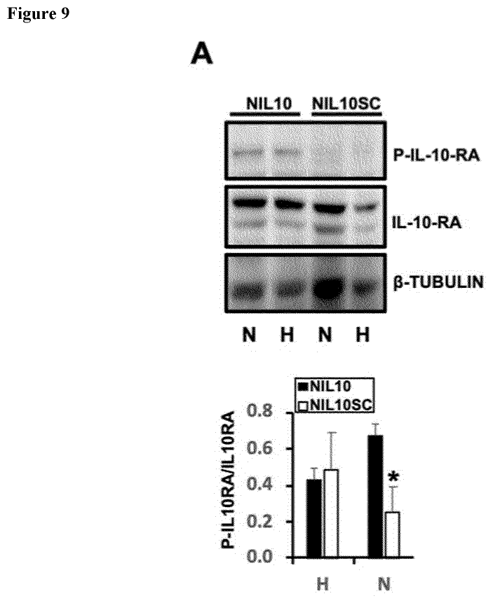

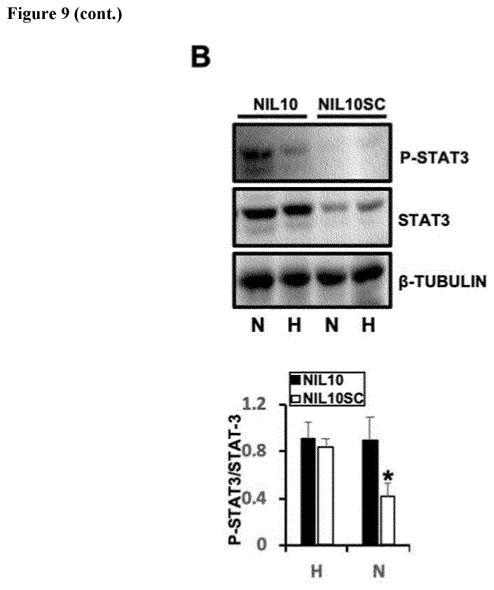

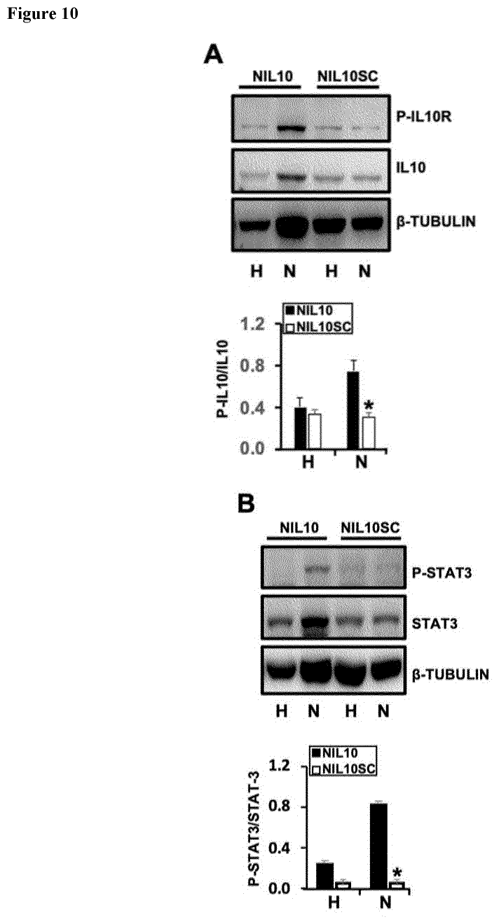

NIL10 led to accumulation of phosphorylated IL-10 receptor subunit IL-10RA and phospho-STAT3 in mice (FIG. 9) and in pigs subjected to IR (FIG. 10), indicative that NIL10, but not NIL10SC, induces receptor-agonist complex activation through the IL10-JNK-STAT-3 signaling pathway.

The anti-inflammatory effect NIL10 was further investigated in RAW 264.7 macrophages stimulated with 500 μM LPS, in which nuclear translocation of pro-inflammatory NF-κB transcription factor was prevented by NIL10 (FIG. 10A), at least by suppressing phosphorylation of the NF-κB cytoplasmic sequestering IκB-α (FIG. 10B), required for IκB-α proteasome-mediated proteolytical degradation.

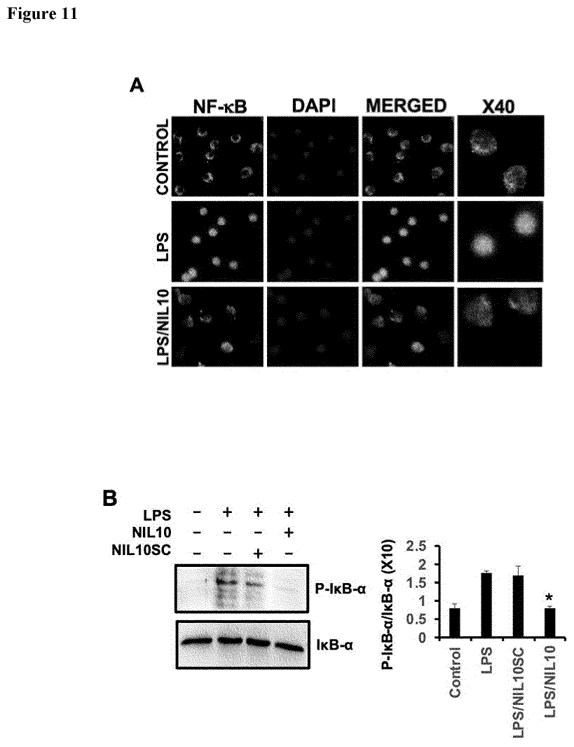

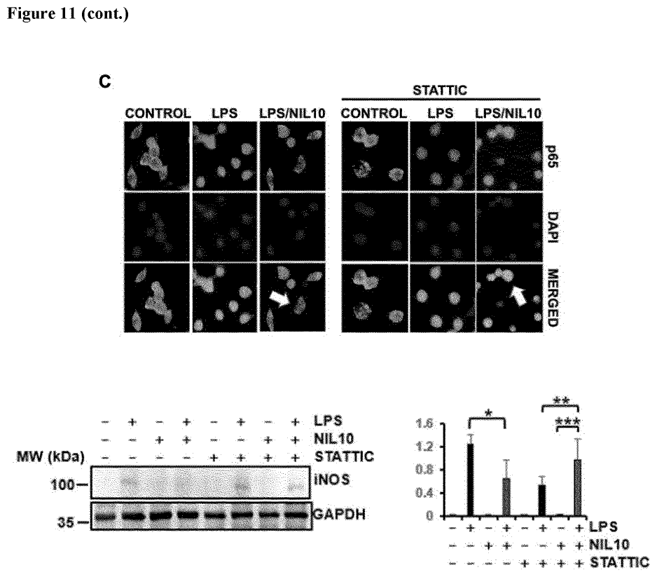

To further validate the anti-inflammatory effect of NIL10, we stimulated NF-κB nuclear translocation with 500 μM LPS in RAW 264.7 cells co-incubated with 5 μM of STAT3-specific pharmacological inhibitor STATTIC, which prevented NIL10-induced cytoplasmic localization of NF-κB in LPS-treated cells, indicating that NIL10 inhibits NF-κB nuclear translocation at least, by STAT-3 activation (FIG. 10C). The expression of the readout inducible nitric oxide synthase (iNOS) by pro-inflammatory signals was also assayed. As shown, iNOS was expressed by stimulating RAW 264.7 macrophages with 500 M LPS, and significantly inhibited by co-incubation with NIL10, while the effect was reversed in the presence of STATTIC (FIG. 11 lower panels). Taken together, NIL10 may act as anti-inflammatory effector through IL-10/STAT3 signaling pathway in myocardial ischemia/reperfusion.

1. A nanoparticle conjugated with a peptide consisting of SEQ ID NO: 1.

2. The nanoparticles of claim 1, further comprising a PEG coating and maleimide linking point, to enhance the pharmacokinetic properties and to enable the linking of SEQ ID NO: 1 peptide, respectively.

3. The nanoparticles of claim 1, further comprising a dye which allows the visualization of the nanoparticle by fluorescence imaging.

4. The nanoparticle of claim 1, further comprising a chemical compound or radioisotopes which allows the detection of the nanoparticle by in vivo molecular imaging techniques.

5. (canceled)

6. A method of preventing and/or treating cardiac damage arising after ischemia followed by reperfusion comprising administering the nanoparticle of claim 1 to a subject in need thereof.

7. A method of preventing and/or treating the inflammatory response following acute myocardial infarction comprising administering the nanoparticle of claim 1 to a subject in need thereof.

8. A pharmaceutical composition comprising the nanoparticle of claim 1, optionally further comprising one or more pharmaceutically acceptable excipients or carriers.

9-10. (canceled)

11. The nanoparticle of claim 1, wherein the size of the nanoparticle is between 100 and 150 nm, the Polydispersity Index (PDI) is lower than 0.3, and the ratio between the total amount of the peptide and the total amount of lipids in the nanoparticle is between 1:5 and 1:15


SEQUENCE LISTING (2 sequences)

SEQ ID NO: 1 - protein, 10 aa, synthetic construct
CAYMTMKIRN

SEQ ID NO: 2 - protein, 10 aa, synthetic construct
CANYRMITKM